SEQUENCE LISTING (3 sequences)

SEQ ID NO: 1 - protein, 1008 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRIRNRSYIDRDSEYLLQENEPDGTLDQKLLEDLQKKKNDLRYIEMQHFREKLPSYGMQKELVNLIDNHQVTVISGETGCGKTTQVTQFILDNYIERGKGSACRIVCTQPRRISAISVAERVAAERAESCGSGNSTGYQIRLQSRLPRKQGSILYCTTGIILQWLQSDPYLSSVSHIVLDEIHERNLQSDVLMTVVKDLLNFRSDLKVILMSATLNAEKFSEYFGNCPMIHIPGFTFPVVEYLLEDVIEKIRYVPEQKEHRSQFKRGFMQGHVNRQEKEEKEAIYKERWPDYVRELRRRYSASTVDVIEMMEDDKVDLNLIVALIRYIVLEEEDGAILVFLPGWDNISTLHDLLMSQVMFKSDKFLIIPLHSLMPTVNQTQVFKRTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVLLSIRHLMELNALDKQEELTPLGVHLARLPVEPHIGKMILFGALFCCLDPVLTIAASLSFKDPFVIPLGKEKIADARRKELAKDTRSDHLTVVNAFEGWEEARRRGFRYEKDYCWEYFLSSNTLQMLHNMKGQFAEHLLGAGFVSSRNPKDPESNINSDNEKIIKAVICAGLYPKVAKIRLNLGKKRKMVKVYTKTDGLVAVHPKSVNVEQTDFHYNWLIYHLKMRTSSIYLYDCTEVSPYCLLFFGGDISIQKDNDQETIAVDEWIVFQSPARIAHLVKELRKELDILLQEKIESPHPVDWNDTKSRDCAVLSAIIDLIKTQEKATPRNFPPRFQDGYYS

SEQ ID NO: 2 - protein, 994 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRIRNRSYIDRDSEYLLQENEPDGTLDQKLLEDLQKKKNDLRYIEMQHFREKLPSYGMQKELVNLIDNHQVTVISGETGCGKTTQVTQFILDNYIERGKGSACRIVCTQPRRISAISVAERVAAERAESCGSGNSTGYQIRLQSRLPRKQGSILYCTTGIILQWLQSDPYLSSVSHIVLDEIHERNLQSDVLMTVVKDLLNFRSDLKVILMSATLNAEKFSEYFGNCPMIHIPGFTFPVVEYLLEDVIEKIRYVPEQKEHRSQFKRGFMQGHVNRQEKEEKEAIYKERWPDYVRELRRRYSASTVDVIEMMEDDKVDLNLIVALIRYIVLEEEDGAILVFLPGWDNISTLHDLLMSQVMFKSVNQTQVFKRTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVLLSIRHLMELNALDKQEELTPLGVHLARLPVEPHIGKMILFGALFCCLDPVLTIAASLSFKDPFVIPLGKEKIADARRKELAKDTRSDHLTVVNAFEGWEEARRRGFRYEKDYCWEYFLSSNTLQMLHNMKGQFAEHLLGAGFVSSRNPKDPESNINSDNEKIIKAVICAGLYPKVAKIRLNLGKKRKMVKVYTKTDGLVAVHPKSVNVEQTDFHYNWLIYHLKMRTSSIYLYDCTEVSPYCLLFFGGDISIQKDNDQETIAVDEWIVFQSPARIAHLVKELRKELDILLQEKIESPHPVDWNDTKSRDCAVLSAIIDLIKTQEKATPRNFPPRFQDGYYS

SEQ ID NO: 3 - protein, 1001 aa, Mus musculus
MSYDYHQSWSRDGGPRGSGQGSSGGGGGGSRGSGGGGGGRGGRGRHPAHLKGREIGLWYAKKQTQKNKEAERQERAVVHMDERREEQIVQLLNSVQAKTDKDSEAQISWFAPEDHGYGTEVSSEKKINSEKKLDNQEKKLLNQEKKTFRITDKSYIDRDSEYLLQENEPNLSLDQHLLEDLQRKKTDPRYIEMQRFRKKLPSYGMQKELVNLINNHQVTVISGETGCGKTTQVTQFILDNYIERGKGSACRIVCTQPRRISAISVAERVATERAESCGNGNSTGYQIRLQSRLPRKQGSILYCTTGIILQWLQSDSRLSSVSHIVLDEIHERNLQSDVLMTVIKDLLHFRSDLKVILMSATLNAEKFSEYFGNCPMIHIPGFTFPVVEYLLEDIIEKIRYVPDQKEHRSQFKRGFMQGHVNRQEKEEKEAIYKERWPAYIKELRTRYSASTVDVLQMMDDDKVDLNLIAALIRYIVLEEEDGAILVFLPGWDNISTLHDLLMSQVMFKSDKFLIIPLHSLMPTVNQTQVFKKTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVVLSIKHLMELSALDKQEELTPLGVHLARLPVEPHIGKMILFGALFCCLDPVLTIAASLSFKDPFVIPLGKEKIADARRKELAKETRSDHLTVVNAFEGWEEAKRRGFRYEKDYCWEYFLSSNTLQMLHNMKGQFAEHLLGAGFVSSRSPKDPKANINSDNEKIIKAVICAGLYPKVAKIRLNLGKKRKMVKVHTKSDGLVSIHPKSVNVEQTDFHYNWLIYHLKMRTSSIYLYDCTEVSPYCLLFFGGDISIQKDKDQEIIAVDEWIVFQSPERIAHLVKGLRKELDSLLQEKIESPHPVDWDDTKSRDCAVLSAILDLIKTQKKATPRNLPPRSQDGYYS

(19) United States
(12) Patent Application Publication
(10) Pub. No.: US 2012/0124682 A1
(43) Pub. Date: May 17, 2012

(54) DHX36 / RHAU KNOCKOUT MICE AS EXPERIMENTAL MODELS OF MUSCULAR DYSTROPHY

(76) Inventors: Janice Lai, Basel (CH); Yoshikuni Nagamine, Riehen (CH); Pu Wu, Shanghai (CN)

(21) Appl. No.: 13/319,279

(22) PCT Filed: May 7, 2010
(86) PCT No.: PCT/EP2010/056226

§ 371 Date: Jan. 25, 2012

(30) Foreign Application Priority Data
May 8, 2009 (EP) 09159803.7

(51) Int. Cl. A01K 67/027; C12N 15/873

(52) U.S. Cl. 800/9; 800/21

(57) ABSTRACT

The present invention provides a genetically-modified non-human animal whose somatic and germ cells contain a gene encoding an altered form of an DHX36 gene, the altered DHX36 haviang been targeted to replace a wild-type DHX36 gene into the animal or an ancestor of the animal at an embyonic stage using embryonic stem cells. An ideal use of the genetically-modified non-human animal of the invention is the use as an experimental model for muscular dystrophy, e.g. spinal muscular atrophy, to identify e.g. new treatments for muscular dystrophy and or study its pathogenesis.

### FIELD OF THE INVENTION

The present invention relates to genetically-modified nonhuman animals useful as an experimental model of muscular dystrophy, wherein the DHX36 gene is altered in said nonhuman animals, producing animals lacking functional DHX36 and developing symptoms of muscular dystrophy.

### BACKGROUND OF THE INVENTION

Muscular dystrophy refers to a group of genetic, hereditary muscle diseases and disorders that cause progressive muscle weakness. These diseases and disorders are characterized by progressive skeletal muscle weakness, defects in muscle proteins, and the death of muscle cells and tissue. Many different diseases including Duchenne, Becker, limb girdle, congenital, facioscapulohumeral, myotonic, oculopharyngeal, distal, and Emery-Dreifuss are classified as muscular dystrophies. Duchenne muscular dystrophy is the most common form of muscular dystrophy and primarily affects boys.

Mutations of the membrane-associated proteins dystrophin and merosin result in progressive muscle wasting and weakness in Duchenne and congenital muscular dystrophy, respectively (Dalkilic, 2003; Tidball, 2005). Boys with Becker muscular dystrophy (very similar to but less severe than Duchenne muscular dystrophy) have faulty or diminished amount of dystrophin. Following muscle plasma membrane damage during contraction, muscle injury produces dys-regulation of a broad spectrum of structural and regulatory genes which accompany muscle fiber death (Petrof et al., 1993; Tidball, 2005; Ge et al., 2004). Interestingly, while initial even severe muscle degeneration results in extremely effective regeneration in cases of rhabdomyolysis, recurrent muscle injury in muscular dystrophy is associated with failure of regeneration and replacement of the muscle tissue with fibrous tissue (fibrosis) and fat.

Spinal Muscular Atrophy (SMA) is a recessively inherited neuromuscular disease characterized by degeneration of spinal cord motor neuron, resulting in progressive muscular atrophy (wasting away) and weakness. The clinical spectrum of SMA ranges from early infant death to normal adult life with only mild weakness. These patients often require comprehensive medical care involving multiple disciplines, including pediatric pulmonology, pediatric neurology, pediatric orthopaedic surgery, pediatric critical care, and physical medicine and rehabilitation; and physical therapy, occupational therapy, respiratory therapy, and clinical nutrition. Spinal muscular atrophy is caused by a deletion or mutation in the survival motor neuron 1 (SMN1) gene. SMN1 was discovered in 1995 and is located on chromosome 5q11-q13.

SMA is characterized by degeneration of motor neurons in the anterior horns of the spinal cord, which leads to progressive symmetrical muscle weakness and atrophy. It affects approximately 1 in 6,000 to 10,000 newborns and previously was the second most common fatal autosomal recessive disorder after cystic fibrosis. Advances in the treatment of cystic fibrosis have resulted in a marked decrease in childhood mortality, such that spinal muscular atrophy is now the leading fatal autosomal recessive disorder in infancy. It is clinically subdivided in 3 types: Type 1 (Werdnig-Hoffmann Disease)is characterized by muscle weakness prior to 6 months of age. Affected infants are unable to sit without support. SMA Type 1 infants account for 60-70% of cases. Prognosis is grave, with most not surviving beyond their second birthday without respiratory support. SMA Type 2 is characterized by symptoms of muscle weakness prior to 18 months of age. Prognosis is widely variable. SMA Type 3 is characterized by the ability to achieve independent ambulation and a normal life expectancy. These patients suffer from proximal muscle weakness, frequent falls, and significant fatigue, yet 40 years after onset, 59% remain ambulatory. Often, these individuals go on to start families of their own. (Source: Spinal Muscular Atrophy Genetic Counseling Access and Genetic Knowledge: Parents' Perspectives by Meldrum, Scott and Swoboda, published in the Journal of Child Neurology, August 2007: http: online sagepub.com)

The term “juvenile spinal muscular atrophy” refers to Kugelberg-Welander syndrome.

In humans and chimpanzees, the region of chromosome 5 that contains the SMN (survival motor neuron) gene has a large duplication. A large sequence that contains several genes occurs twice—i.e. once in each of the adjacent segments. A second change that is found only in humans is that the two copies of the gene—known as SMN1 and SMN2—differ by only a few base pairs. The important change in the SMN2 gene, for the purposes of SMA, is a silent mutation that occurs at the splice junction of intron 6 to exon 7. This affects splicing of the SMN2 pre-RNA, resulting in about 90% of the transcripts being inappropriately spliced into a form that excludes exon 7. This shorter mRNA transcript codes for a shorter SMN protein, which is rapidly degraded. About 10% of the mRNA transcript from SMN2 is spliced into the full length transcript that codes for the fully functional SMN protein.

SMA is caused by loss of the SMN1 gene from both chromosomes. The severity of SMA, ranging from SMA 1 to SMA 3, is partly related to how well the remaining SMN 2 genes can make up for the loss of SMN 1. In part this is related to how many copies of the SMN2 gene are present. The mutations that cause the loss of SMN 1 are of two types. One is a deletion mutation, decreasing the copy number of SMN1 by one. The other is a conversion mutation (rare in animals), that changes the SMN 1 sequence to that of SMN2. By this and other means, additional copies of SMN 2 may arise. Two copies is most often associated with SMA type 1. There is a lot of overlap between the type of SMA and the number of copies, however, such that copy number is not useful to determine the type of SMA any one individual has. A much rarer form of severe infantile form of SMA, exclusively affecting boys, is caused by missense and synonymous variants mutations in gene UBE1 on the X chromosome. Sons of women with this genetic mutation generally have a 50% chance of suffering the disease, and daughters of women with the mutation have a 50% chance of being carriers, while unaffected by the disease itself. All forms of SMN-associated SMA have a combined incidence of about 1 in 6,000. SMA is the most common cause of genetically determined neonatal death. The gene frequency is thus around 1:80, and thus approximately one in 40 persons are carriers.

### SUMMARY OF THE INVENTION

The present inventors have now surprisingly found that the inhibition in mice of DHX36, which was initially cloned by some of the inventors, leads to symptoms which are similar to the ones observed in human muscular dystrophy, hence providing an extremely useful experimental model to study the mechanisms leading to muscular dystrophy.

The present invention hence provides a genetically-modified non-human animal whose somatic and germ cells contain a gene encoding an altered form of an DHX36 gene, the altered DHX36 gene having been targeted to replace a wild-type DHX36 gene into the animal or an ancestor of the animal at an embryonic stage using embryonic stem cells. In some embodiments of the invention, the genetically-modified non-human animal is a mouse.

In some embodiments of the invention, the genetically-modified non-human animal is fertile and is capable of transmitting the altered DHX36 gene to its offspring.

For example, the altered DHX36 gene can be introduced into an ancestor of the genetically-modified non-human animal of the invention at an embryonic stage by electroporation of altered embryonic stem cells. In some embodiments, the altered DHX36gene can be introduced into the genetically-modified non-human animal at an embryonic stage either by electroporation of altered embryonic stem cells into genetically-modified non-human animal blastocysts or co-incubation of altered embryonic stem cells with fertilized eggs or morulae.

The altered form of DHX36can be, ins some embodiments of the invention, either nonfunctional or is derived from a species other than said genetically-modified non-human animal, for instance human DHX36.

An ideal use of the genetically-modified non-human animal of the invention is the use as an experimental model for muscular dystrophy, e.g. spinal muscular atrophy, to identify e.g. new treatments for muscular dystrophy, e.g. spinal muscular atrophy, and or study its pathogenesis.

The present invention also provides methods of producing a genetically-modified non-human animal whose somatic and germ cells contain a gene encoding an altered form of DHX36. In some embodiments, the altered gene can have been targeted to replace the wild-type DHX36 gene into the genetically-modified non-human animal or an ancestor of said genetically-modified non-human animal at an embryonic stage using embryonic stem cells by a method comprising the steps of (i) introducing a gene encoding an altered form of DHX36 designed to target the DHX36 gene into embryonic stem cells of said genetically-modified non-human animal, for example by electroporation, (ii) injecting the embryonic stem cells containing the altered DHX36 gene into blastocysts of said genetically-modified non-human animal, (iii) transplanting the injected blastocysts into a recipient genetically-modified non-human animal; and (iv) allowing the embryo to develop producing a founder genetically-modified non-human animal mouse.

### BRIEF DESCRIPTION OF THE DRAWINGS

FIG. 1: RHAU^{fl/fl} mice were prepared by a standard gene targeting method, in which loxP sites are inserted at both sides of exon 8 of the RHAU gene.

FIG. 2: Confirmation of correct integration of targeting vector by southern blot. DNA samples from ES cells were digested with restriction enzyme, AspI. One AspI site is introduced into the genome when recombination of targeting vector occured. Internal and external probe were used to identify the clones with correct recombination of the targeting vector. Correct recombination would give three bands, 15 kb, 10.7 kb and 6.5 kb if an internal probe is used. The 15 kb fragment is corresponding to the wildtype allele and present of both 10.7 and 6.5 kb bands represent the transgenic allele. Two bands, 15 kb and 10.7 kb, were detected when the external probe was used. The 15 kb band corresponds to the wildtype allele and the 10.7 kb band corresponds to the transgenic allele. Clone 42, 2 52, 63 and 208 showed correct integration of the targeting vector.

FIG. 3: After 4 weeks of tamoxifen treatment, i.e. of disruption of RHAU (DHX36) expression, all RHAU ^{fl/fl}; Cre-ER™ mice (A) hold their hindlimbs into their body, whereas the control RHAU^{fl/fl} littermate (B) spread its limbs outwards, as normal mice do when their tail is hold and they are suspended over their cage.

FIG. 4: After 10 weeks of tamoxifen treatment, i.e. of disruption of RHAU (DHX36) expression, RHAU fl/fl; Cre-ER™ mice show a progressing muscular dystrophy phenotype. Note the marked paralysis with abnormal posture of the limbs and cyphosis of RHAU fl/fl; Cre-ER™ (C), as compared to the RHAU fl/fl control littermate (D)

FIG. 5: After 10 weeks of tamoxifen treatment, i.e. of disruption of RIIAU (DHX36) expression, the relative size of skeletal muscle to the size of the hindlimb bone of RHAU^{fl/fl}; Cre-ER™ mice (Left) is significantly smaller than in the RHAU^{fl/fl}(Right) control littermates.

### DETAILED DESCRIPTION OF THE INVENTION

The present inventors have now surprisingly found that the inhibition in mice of DHX36, a protein which was initially cloned by some of the inventors, leads to symptoms which are similar to the ones observed in human muscular dystrophy.

The present invention hence provides a genetically-modified non-human animal whose somatic and germ cells contain a gene encoding an altered form of an DHX36 gene, the altered DHX36 gene having been targeted to replace a wild-type DHX36 gene into the animal or an ancestor of the animal at an embryonic stage using embryonic stem cells. In some embodiments of he invention, the genetically-modified non-human animal is a mouse.

In some embodiments of the invention, the genetically-modified non-human animal is fertile and is capable of transmitting the altered DHX36 gene to its offspring.

For example, the altered DHX36 gene can be introduced into an ancestor of the genetically-modified non-human animal of the invention at an embryonic stage by electroporation of altered embryonic stem cells. In some embodiments, the altered DHX36 gene can be introduced into the genetically-modified non-human animal at an embryonic stage either by electroporation of altered embryonic stem cells into genetically-modified non-human animal blastocysts or coincubation of altered embryonic stem cells with fertilized eggs or morulae.

The altered form of DHX36 can be, in some embodiments of the invention, either nonfunctional or is derived from a species other than said genetically-modified non-human animal, for instance human DHX36.

An ideal use of the genetically-modified non-human animal of the invention is the use as an experimental model for muscular dystrophy, to identify e.g. new treatments for a muscular dystrophy, e.g. spinal muscular atrophy, and or study the pathogenesis of this disease.

The present invention also provides methods of producing a genetically-modified non-human animal whose somatic and germ cells contain a gene encoding an altered form of DHX36. In some embodiments, the altered gene can have been targeted to replace the wild-type DHX36 gene into the genetically-modified non-human animal or an ancestor of said genetically-modified non-human animal at an embryonic stage using embryonic stem cells by a method comprising the steps of (i) introducing a gene encoding an altered form of DHX36 designed to target the DHX36 gene into embryonic stem cells of said genetically-modified non-human animal, for example by electroporation, (ii) injecting the embryonic stem cells containing the altered DHX36 gene into blastocysts of said genetically-modified non-human animal, (iii) transplanting the injected blastocysts into a recipient genetically-modified non-human animal; and (iv) allowing the embryo to develop producing a founder genetically-modified non-human animal mouse.

Another embodiment of the invention encompasses the use of the DHX36 gene or DHX36 protein as a biomarker for muscular dystrophy, for instance by measuring the concentration of this protein, or the expression levels of the gene, in samples from a subject.

In some embodiments of the present invention, the DHX36 gene clone and the corresponding locus in the genome are used to generate genetically-modified animals in which the DHX36 gene has been made non-functional. The alterations to the naturally occurring gene can be modifications, deletions and substitutions. Modifications and deletions render the naturally occurring gene nonfunctional, producing a “knockout” animal. Substitution of the naturally occurring gene for a gene from a second species results in an animal which produces the gene product of the second species. Substitution of the naturally occurring gene for a gene having a mutation results in an animal which produces the mutated gene product. These genetically-modified animals are critical for therapeutic drug studies, the creation of animal models of human diseases, and for eventual treatment of disorders or diseases associated with human homologue of the DHX36 family as described herein and elsewhere. A genetically-modified animal carrying a disruption or “knockout” of the DHX36 gene is useful for the establishment of a non-human model for diseases involving DHX36, for instance muscular dystrophy.

The term “non-human animal” is used herein to include all vertebrate animals, except humans. Examples of non-human animals are mice, rats, cows, pigs, horses, chickens, ducks, geese, cats, dogs, etc. The term “non-human animal” also includes an individual animal in all stages of development, including embryonic and fetal stages. A “genetically-modified animal” is any animal containing one or more cells bearing genetic information altered or received, directly or indirectly, by deliberate genetic manipulation at a sub-cellular level, such as by targeted recombination, microinjection or infection with recombinant virus. The term “genetically-modified animal” is not intended to encompass classical crossbreeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA. The term “germ-line genetically-modified animal” refers to a genetically-modified animal in which the genetic alteration or genetic information was introduced into germline cells, thereby conferring the ability to transfer the genetic information to its offspring. If such offspring in fact possess some or all of that alteration or genetic information, they are genetically-modified animals as well.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene, or not expressed at all.

The non-functional DHX36 gene generally should not fully encode the same DHX36 native to the host animal, and its expression product should be altered to a minor or great degree, or absent altogether. However, it is conceivable that a more modestly modified DHX36 will fall within the scope of the present invention.

The genes used for altering a target gene may be obtained by a wide variety of techniques that include, but are not limited to, isolation from genomic sources, preparation of cDNAs from isolated mRNA templates, direct synthesis, or a combination thereof.

A type of target cells for transgene introduction is the ES cells. ES cells may be obtained from pre-implantation embryos cultured in vitro and fused with embryos (Evans et al. (1981), Nature 292:154-156; Bradley et al. (1984), Nature 309:255-258; Gossler et al. (1986), Proc. Natl. Acad. Sci. USA 83:9065-9069; Robertson et al. (1986), Nature 322:445-448; Wood et al. (1993), Proc. Natl. Acad. Sci. USA 90:4582-4584). Transgenes can be efficiently introduced into the ES cells by standard techniques such as DNA transfection using electroporation or by retrovirus-mediated transduction. The resultant transformed ES cells can thereafter be combined with morulas by aggregation or injected into blastocysts from a non-human animal. The introduced ES cells thereafter colonize the embryo and contribute to the germline of the resulting chimeric animal (Jaenisch (1988), Science 240:1468-1474). The use of gene-targeted ES cells in the generation of gene-targeted genetically-modified mice was described 1987 (Thomas et al. (1987), Cell 51:503-512) and is reviewed elsewhere (Frohman et al. (1989), Cell 56:145-147; Capecchi (1989), Trends in Genet. 5:70-76; Baribault et al. (1989), Mol. Biol. Med. 6:481-492; Wagner (1990), EMBO J. 9:3025-3032; Bradley et al. (1992), Bio/Technology 10:534-539).

Techniques are available to inactivate or alter any genetic region to any mutation desired by using targeted homologous recombination to insert specific changes into chromosomal alleles.

As used herein, a “targeted gene” is a DNA sequence introduced into the germline of a non-human animal by way of human intervention, including but not limited to, the methods described herein. The targeted genes of the invention include DNA sequences which are designed to specifically alter cognate endogenous alleles.

As used herein “treating” includes achieving, partially or substantially, one or more of the following results: partially or totally reducing the extent of the disease, disorder or syndrome; ameliorating or improving a clinical symptom or indicator associated with the disorder; delaying, inhibiting or preventing the progression of the disease, disorder or syndrome; or partially or totally delaying, inhibiting or preventing the onset or development of disorder. Delaying, inhibiting or preventing the progression of the disease, disorder or syndrome includes for example, delaying, inhibiting or preventing muscular dystrophy.

The present invention also provides a method of screening compounds to identify those which might be useful for treating muscular dystrophy in a subject, for instance by modulating the expression or the protein levels of DHX36, as well as the so-identified compounds.

The present invention hence also provides pharmaceutical compositions for treating muscular dystrophy in a subject, for instance by modulating the expression or the protein levels of DHX36. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachet indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single-and double-stranded DNA, DNA that is a mixture of single-and double-stranded regions, single-and double-stranded RNA, and RNA that is mixture of single- and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U′s to T′s. The result of said global sequence alignment is in percent identity. Preferred parameters used in a FASTDB alignment of DNA sequences to calculate percent identity are: Matrix=Unitary, k-tuple=4, Mismatch Penalty--1, Joining Penalty--30, Randomization Group Length=0, Cutoff Score=l, Gap Penalty--5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty--I, Joining Penalty=20, Randomization Group Length=0, Cutoff Score=I, Window Size=sequence length, Gap Penalty--5, Gap Size Penalty--0.05, Window Size=500 or the length of the subject amino acid sequence, whichever is shorter. If the subject sequence is shorter than the query sequence due to N-or C-terminal deletions, not because of internal deletions, a manual correction must be made to the results. This is because the FASTDB program does not account for N-and C-terminal truncations of the subject sequence when calculating global percent identity. For subject sequences truncated at the N-and C-termini, relative to the query sequence, the percent identity is corrected by calculating the number of residues of the query sequence that are N-and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N-and C-termini of the subject sequence, which are not matched/aligned with the query sequence, are considered for the purposes of manually adjusting the percent identity score. That is, only query residue positions outside the farthest N-and C-terminal residues of the subject sequence. Only residue positions outside the N-and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N-or C-terminal residues of a protein retains such immunogenic activities can readily be determined by routine methods described herein and otherwise known in the art.

Muscular dystrophies encompass a group of inherited, progressive muscle disorders, distinguished clinically by the selective distribution of skeletal muscle weakness. The two most common forms of muscle dystrophy are Duchenne and Becker dystrophies, each resulting from the inheritance of a mutation in the dystrophin gene, which is located at the Xp21 locus. Other dystrophies include, but are not limited to, limb-girdle muscular dystrophy which results from mutation of multiple genetic loci including the p94 calpain, adhalin, y-sarcoglycan, and (3-sarcoglycan loci; fascioscapulohumeral (Landouzy-Dejerine) muscular dystrophy, myotonic dystrophy, and Emery-Dreifuss muscular dystrophy. The symptoms of Duchenne muscular dystrophy, which occurs almost exclusively in males, include a waddling gait, toe walking, lordosis, frequent falls, and difficulty in standing up and climbing stairs. Symptoms start at about 3-7 years of age with most patients confined to a wheelchair by 10-12 years and many die at about 20 years of age due to respiratory complications. Current treatment for Duchenne muscular dystrophy includes administration of prednisone (a corticosteroid drug), which while not curative, slows the decline of muscle strength and delays disability. Corticosteroids, such as prednisone, are believed to act by blocking the immune cell activation and infiltration which are precipitated by muscle fiber damage resulting from the disease. Unfortunately, corticosteroid treatment also results in skeletal muscle atrophy which negates some of the potential benefit of blocking the immune response in these patients. Thus, there is a continuing need to identify therapeutic agents which slow the muscle fiber damage and delay the onset of disability in patients with muscular dystrophies, but cause a lesser degree of skeletal muscle atrophy than current therapies.

For the purpose of the present invention, spinal muscular atrophy is considered to be a muscular dystrophy. Spinal Muscular Atrophy (SMA) is a recessively inherited neuromuscular disease characterized by degeneration of spinal cord motor neuron, resulting in progressive muscular atrophy (wasting away) and weakness. The clinical spectrum of SMA ranges from early infant death to normal adult life with only mild weakness. These patients often require comprehensive medical care involving multiple disciplines, including pediatric pulmonology, pediatric neurology, pediatric orthopaedic surgery, pediatric critical care, and physical medicine and rehabilitation; and physical therapy, occupational therapy, respiratory therapy, and clinical nutrition. Spinal muscular atrophy is caused by a deletion or mutation in the survival motor neuron 1 (SMN1) gene. SMN1 was discovered in 1995 and is located on chromosome 5q11-q13. SMA is characterized by degeneration of motor neurons in the anterior horns of the spinal cord, which leads to progressive symmetrical muscle weakness and atrophy. It affects approximately 1 in 6,000 to 10,000 newborns and previously was the second most common fatal autosomal recessive disorder after cystic fibrosis. Advances in the treatment of cystic fibrosis have resulted in a marked decrease in childhood mortality, such that spinal muscular atrophy is now the leading fatal autosomal recessive disorder in infancy. It is clinically subdivided in 3 types: Type 1 (Werdnig-Hoffmann Disease)is characterized by muscle weakness prior to 6 months of age. Affected infants are unable to sit without support. SMA Type 1 infants account for 60-70% of cases. Prognosis is grave, with most not surviving beyond their second birthday without respiratory support. SMA Type 2 is characterized by symptoms of muscle weakness prior to 18 months of age. Prognosis is widely variable. SMA Type 3 is characterized by the ability to achieve independent ambulation and a normal life expectancy. These patients suffer from proximal muscle weakness, frequent falls, and significant fatigue, yet 40 years after onset, 59% remain ambulatory. Often, these individuals go on to start families of their own. (Source: Spinal Muscular Atrophy Genetic Counseling Access and Genetic Knowledge: Parents' Perspectives by Meldrum, Scott and Swoboda, published in the Journal of Child Neurology, August 2007: http: online sagepub.com)

The term “juvenile spinal muscular atrophy” refers to Kugelberg-Welander syndrome.

In humans and chimpanzees, the region of chromosome 5 that contains the SMN (survival motor neuron) gene has a large duplication. A large sequence that contains several genes occurs twice—i.e. once in each of the adjacent segments. A second change that is found only in humans is that the two copies of the gene—known as SMN1 and SMN2—differ by only a few base pairs. The important change in the SMN2 gene, for the purposes of SMA, is a silent mutation that occurs at the splice junction of intron 6 to exon 7. This affects splicing of the SMN2 pre-RNA, resulting in about 90% of the transcripts being inappropriately spliced into a form that excludes exon 7. This shorter mRNA transcript codes for a shorter SMN protein, which is rapidly degraded. About 10% of the mRNA transcript from SMN2 is spliced into the full length transcript that codes for the fully functional SMN protein.

SMA is caused by loss of the SMN1 gene from both chromosomes. The severity of SMA, ranging from SMA 1 to SMA 3, is partly related to how well the remaining SMN 2 genes can make up for the loss of SMN 1. In part this is related to how many copies of the SMN2 gene are present. The mutations that cause the loss of SMN 1 are of two types. One is a deletion mutation, decreasing the copy number of SMN1 by one. The other is a conversion mutation (rare in animals), that changes the SMN 1 sequence to that of SMN2. By this and other means, additional copies of SMN 2 may arise. Two copies is most often associated with SMA type 1. There is a lot of overlap between the type of SMA and the number of copies, however, such that copy number is not useful to determine the type of SMA any one individual has. A much rarer form of severe infantile form of SMA, exclusively affecting boys, is caused by missense and synonymous variants mutations in gene UBE1 on the X chromosome. Sons of women with this genetic mutation generally have a 50% chance of suffering the disease, and daughters of women with the mutation have a 50% chance of being carriers, while unaffected by the disease itself. All forms of SMN-associated SMA have a combined incidence of about 1 in 6,000. SMA is the most common cause of genetically determined neonatal death. The gene frequency is thus around 1:80, and thus approximately one in 40 persons are carriers.

DEAH (Asp-Glu-Ala-His) box polypeptide 36 (EC 3.6.1.-) is also known as “DHX36”. Additional alternative names of “DHX36” are G4 resolvase-1 (G4R1), RNA helicase associated with AU-rich element (RHAU), DEAH box protein 36 (KIAA1488), MLE-like protein 1 (MLEL1), or DEAD/H (Asp-Glu-Ala-Asp/His) box polypeptide 36 (DDX36). This gene is a member of the DEAH-box family of RNA-dependent NTPases which are named after the conserved amino acid sequence Asp-Glu-Ala-His in motif II. The protein encoded by this gene has been shown to enhance the deadenylation and decay of mRNAs with 3′-UTR AU-rich elements (ARE-mRNA). The protein has also been shown to resolve into single strands the highly stable tetramolecular DNA configuration (G4) that can form spontaneously in guanine-rich regions of DNA. Alternative splicing results in multiple transcript variants encoding different isoforms. DEAD box proteins, characterized by the conserved motif Asp-Glu-Ala-Asp (DEAD), are putative RNA helicases. They are implicated in a number of cellular processes involving alteration of RNA secondary structure such as translation initiation, nuclear and mitochondrial splicing, and ribosome and spliceosome assembly. Based on their distribution patterns, some members of this DEAD box protein family are believed to be involved in embryogenesis, spermatogenesis, and cellular growth and division. The Entrez GeneID for DHX36-1 is: 170506. The amino acid sequence of the two known human isoforms (isoform 1 and isoform 2) of DHX36 as well as the amino acid sequence of the mouse DHX36 can be found in the sequence listing (SEQ ID:1, 2 and 3, respectively).

The present invention also provides a method of screening compounds to identify those which might be useful for treating cancer in a subject by inhibiting DHX36 as well as the so-identified compounds.

In some embodiment of the invention, the genetically-modified non-human animal is a conditional DHX36 knockout, for instance a “foxed” conditional knockout mouse established using the loxP/Cre recombinase system. The loxP/Cre recombinase system is well-known in the art. As used herein, “conditional knockout” refers to a genetically modified non-human organism that has a genome, in which a particular gene has been disrupted or deleted such that expression of the gene is eliminated or occurs at a reduced level in a specific cell type or tissue (Kwan, Genesis, 32, 49-62 (2002)) (Rajewsky, et al., J Clin Invest, 98, 600-603 (1996)). The disruption or deletion of the particular gene, in this case the DHX36 gene, is based on the interaction of the following elements: loxP-sites in the DHX36 gene and Cre-Recombinase under the control of a tissue specific promoter. The transgenic, conditional knockout mouse of the invention lacks a functional DHX36 gene product or exhibits a reduced level of the DHX36 gene product. The mutant non-human animal is referred to hereinafter as a “conditional DHX36 knockout non-human animal” or “floxed DHX36 knockout non-human animal”. The present invention also encompasses methods of producing a transgenic non-human animal that lacks a functional DHX36 gene in a conditional manner Briefly, the standard methodology for producing a conditional knockout animal is well known in the art (Kwan, Genesis, 32, 49-62 (2002)) (Rajewsky, et al., J Clin Invest, 98, 600-603 (1996)) and requires the crossing of an allele of the target gene, that has been modified by the insertion of two Cre recombinase recognition (loxP) sequences within intron regions (“floxed”), to a second mouse strain that expresses Cre recombinase in a specific cell type or tissue. By the action of Cre, the loxP flanked gene segment is excised and deleted from the genome leading to the inactivation of the DHX36 gene.

As a result of the conditional disruption of the DHX36 gene, the DHX36 conditional knockout mouse of the present invention manifests a particular phenotype. The term “phenotype” refers to the resulting biochemical, physiological or behavioral consequences attributed to a particular genotype. In the situation where a conditional knockout mouse has been created, the phenotype observed is a result of the loss of the gene that has been knocked out. In the mice generated in the following examples, a loss of the DHX36 protein is observed. Without wishing to be bound by theory, the phenotype observed, might be a result of a primary degeneration of the neurons innervating the muscles. Such transgenic animals are well suited for, e.g., pharmacological studies of drugs.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Generation of RHAU fl/fl mice

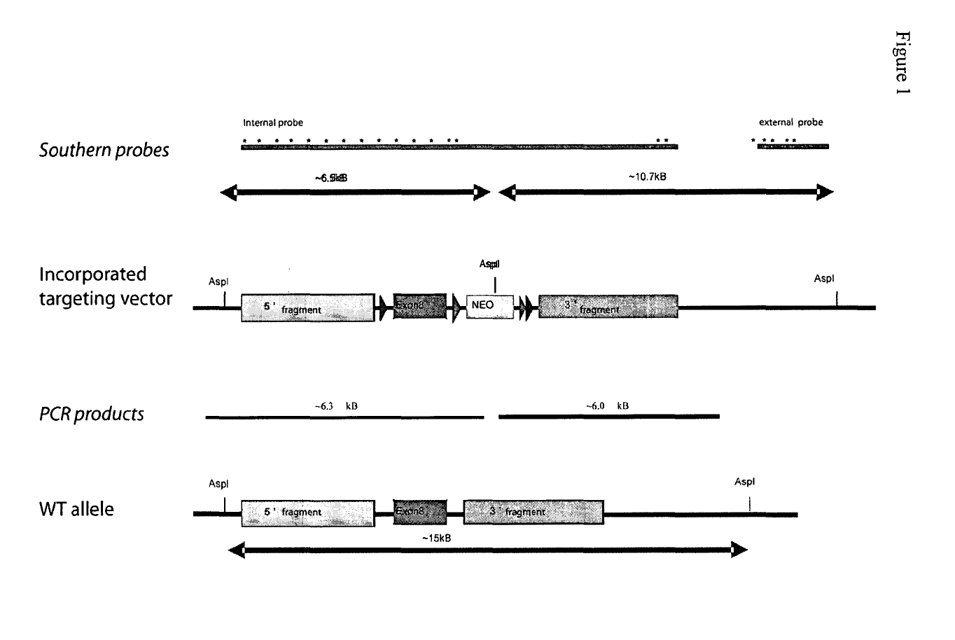

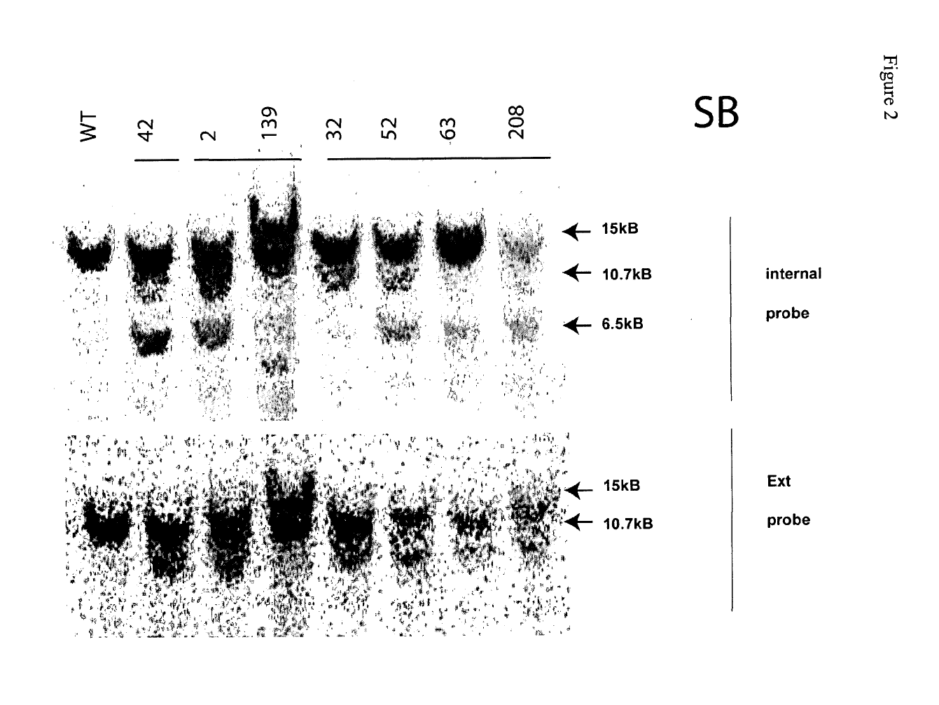

RHAU^{fl/fl} mice were prepared by a standard gene targeting method, in which loxP sites are inserted at both sides of exon 8 of the RHAU gene, as illustrated in FIG. 1.

In order to remove the neomycin cassette, floxed mice with neomycin cassette were crossed with 129S4/SvJaeSor-Gt(ROSA)26Sor^{tm1(FLP1)Dym}/J mice from the Jackson Laboratory which contain FLP1 transgene and express FLP1 recombinase (FLP^{tg}). F1 offspring RHAU^{fl/+}; FLP^{tg} were then crossed with F1 RHAU^{fl/+}; FLP^{tg} mice in order to get homologous floxed mice without neomycin cassette but negative of FLP1 transgene. After this stage, the RHAU^{fl/fl} mice no longer contain the neomycin cassette. These new RHAU^{fl/fl} mice then were crossed with B6.Cg-Tg(CAG-cre/Esr1)5Amc/J from the Jackson Laboratory (Cre-ER™) which express a fusion protein between Cre and a mutated form of the ligand binding domain of the estrogen receptor (Cre-ER™) that confers them a tamoxifen™ inducible Cre activity. Offspring with genotype RHAU^{fl/+}; Cre ER™ mice were crossed with RHAU^{fl/fl} or RHAU^{fl/+} mice to obtain RHAU^{fl/fl}; Cre-ER™ for experiments using RHAU^{fl/fl} littermates as controls.

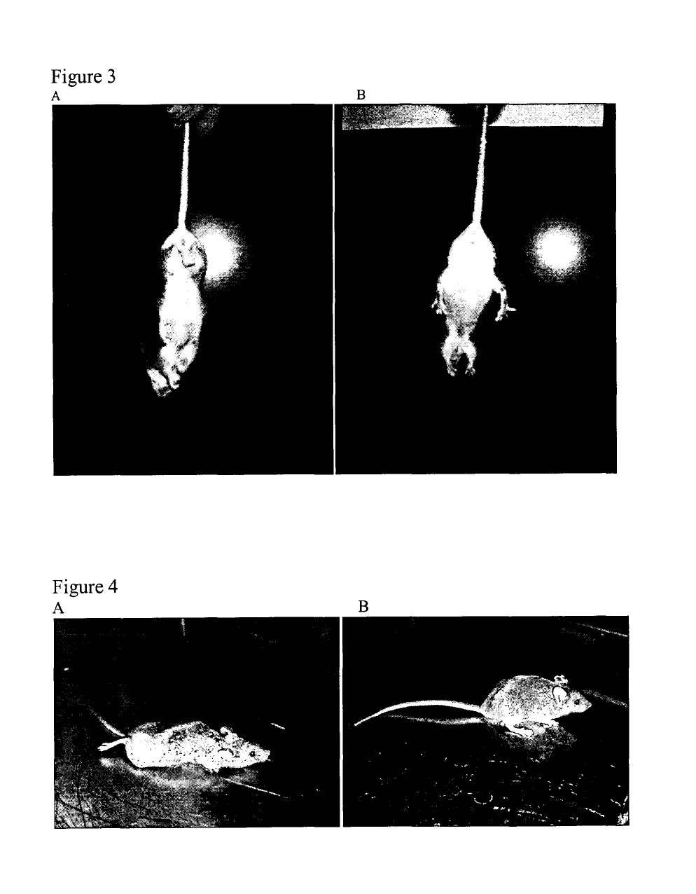

Two to five months old mice with genotypes RHAU^{fl/fl}; Cre-ER™ and their littermate mice with genotype RHAU^{fl/fl} were injected with 10 doses of 100 mg/kg tamoxifen (Sigma, T 5648; 1 g of tamoxifen was dissolved in 10 ml of ethanol at 42° C. and further diluted with 90 ml of corn oil to give a working stock 10 mg/ml.). After 4 weeks, all RHAU^{fl/fl}; Cre-ER™ mice held their hindlimbs into their body (FIG. 3A), whereas the control RHAU^{fl/fl}littermate spread its limbs outwards (FIG. 3B), as mice usually do when their tail were hold and suspended over the cage. After 10 weeks of the tamoxifen treatment, RHAU^{fl/fl}; Cre-ER™ mice show progressing phenotype of muscular dystrophy (FIG. 4). Note the marked paralysis with abnormal posture of the limbs and cyphosis of RHAU^{fl/fl}; Cre-ER™ (FIG. 4A) compare to the RHAU^{fl/fl} control littermate (FIG. 4B).

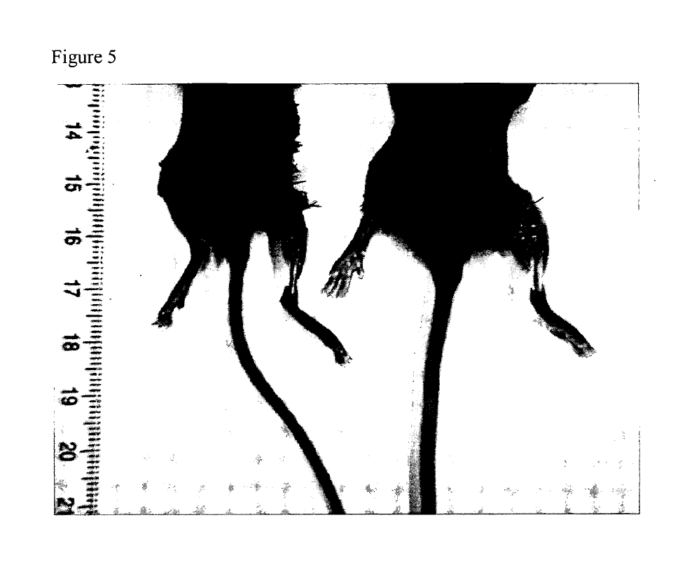

After 10 weeks of the first dosage of tamoxifen treatment, the relative size of skeletal muscle to the size of of the hindlimb bone of RHAU fl/fl; Cre-ET™ mice (Left) was significantly smaller than in the RHAU fl/fl (Right) control littermates (FIG. 5).

1. A genetically-modified non-human animal whose somatic and germ cells contain a gene encoding an altered form of a DHX36 gene, the altered DHX36 gene having been targeted to replace a wild-type DHX36 gene into the animal or an ancestor of the animal at an embryonic stage using embryonic stem cells.

2. The genetically-modified non-human animal of claim 1 wherein said animal is a mouse.

3. The genetically-modified non-human animal of any of claim 1, wherein said genetically-modified non-human animal is fertile and capable of transmitting the altered DHX36 gene to its offspring.

4. The genetically-modified non-human animal of claim 1, wherein the altered DHX36 gene has been introduced into an ancestor of the genetically-modified non-human animal at an embryonic stage by electroporation of altered embryonic stem cells.

5. The genetically-modified non-human animal of claim 1, wherein the altered DHX36 gene has been introduced into the genetically-modified non-human animal at an embryonic stage either by electroporation of altered embryonic stem cells into genetically-modified non-human animal blastocysts or coincubation of altered embryonic stem cells with fertilized eggs or morulae.

6. The genetically modified animal of claim 1, wherein said altered form of DHX36 is either nonfunctional or is derived from a species other than said genetically-modified non-human animal.

7. The genetically-modified non-human animal of claim 1, wherein said altered form of DHX36 is human DHX36.

8. (canceled)

9. (canceled)

10. The genetically-modified non-human animal of claim 1, wherein the altered form of a DHX36 gene is only expressed or removed upon addition of a further component.

11. The genetically-modified non-human animal of claim 10, wherein said altered form of a DHX36 gene consists of the wild-type DHX36 gene placed between two fox loci and the further component is a CRE recombinase.

12. A method of producing a genetically-modified non-human animal whose somatic and germ cells contain a gene encoding an altered form of DHX36, the altered gene having been targeted to replace the wild-type DHX36 gene into the genetically-modified non-human animal or an ancestor of said genetically-modified non-human animal at an embryonic stage using embryonic stem cells, which comprises:
introducing a gene encoding an altered form of DHX36 designed to target the DHX36 gene into embryonic stem cells of said genetically-modified non-human animal;
injecting the embryonic stem cells containing the altered DHX36 gene into blastocysts of said genetically-modified non-human animal;
transplanting the injected blastocysts into a recipient genetically-modified non-human animal; and
allowing the embryo to develop producing a founder genetically-modified non-human animal.

13. (canceled)

14. (canceled)

15. (canceled)
